(19) United States
(12) Patent Application Publication
(10) Pub. No.: US 2002/0128320 A1
(43) Pub. Date: Sep. 12, 2002

(54) Method of administering calcium citrate

(75) Inventor: Ian R. Reid, Auckland (NZ)

Correspondence Address: FULBRIGHT & JAWORSKI, LLP, 1301 MCKINNEY, SUITE 5100, HOUSTON, TX, 77010-3095, US

(73) Assignee: UniServices Ltd.

(21) Appl. No.: 10/016,371

(22) Filed: Dec. 10, 2001

(51) Int. Cl. A61K 31/19

(52) U.S. Cl. 514/574

(57) ABSTRACT

The present invention describes a method of increasing the high-density lipoprotein level in plasma by administering to a postmenopausal woman a pharmaceutical formulation comprised of calcium citrate. The invention also provides for a pharmaceutical composition and a dietary supplement thereof.

### BACKGROUND OF THE INVENTION

1. Field of the Invention

The invention is related to the field of medicine and physiology and pertains to the administration of calcium citrate to improve serum lipid levels in a postmenopausal woman.

2. Related Art

Calcium forms hydroxyapatite, the major mineral constituent of bone, and is an essential element in many biological processes, including mediating some hormonal signals, triggering muscle contraction, transmitting nerve impulses and blood clotting. As a consequence, calcium is vital to normal bone formation and maintenance of bone density. Long-term dietary calcium insufficiency almost always results in net loss of calcium from the bones. The U.S. recommended daily allowance (RDA) for calcium is 800 mg/day, with an additional 400 mg advisable for pregnant and lactating women. A large proportion of the over-60 population consumes less than one-half of the RDA for calcium. This is also the age group most at risk of developing osteoporosis, which is characterized by loss of the organic matrix as well as progressive demineralization of the bone. Calcium supplementation is widely recommended and used amongst postmenopausal women for prevention of osteoporosis (Genant et al., 1999), and consistent evidence from randomized controlled trials have demonstrated that calcium supplementation slows postmenopausal bone loss (Dawson-Hughes, et al., 1990; Reid et al., 1993). There is also some evidence that calcium supplementation prevents fractures in postmenopausal women (Chevalley et al., 1994; Reid et al., 1995; Recker et al., 1996). Other benefits from the use of calcium supplements have been suggested, including effects on colon cancer, blood pressure and serum lipids (Barger-Lux et al., 1994).

The suggestion that calcium intakes impacts serum lipid concentrations has arisen from human and animal studies which indicate that calcium binds to fatty acids and bile acids in the gut, thereby interfering with their absorption (Govers et al., 1993; Denke et al., 1993; Mitchell et al., 1968). However, these suggestions are counter-balanced by studies showing no effect of calcium supplementation on circulating lipid concentrations (Mitchell et al., 1968; Bostick et al., 2000). As a result, the entire span of health benefits afforded by calcium supplementation has remained unrealized. There has been no long-term randomized controlled trial to address the many discrepancies, including no adequate studies in normal postmenopausal women, the group most commonly using calcium supplementation.

Previous investigations on the effect of calcium on serum lipids involved primarily the administration of calcium carbonate either singularly or in conjunction with other nutritional supplements despite the reported side effects of constipation and abdominal distress (Yacowitz et al., 1965; Carlson et al. 1971; Bhattacharyya et al., 1969; Bierenbaum et al., 1972; Albanese et al., 1973; Bell et al., 1992). Calcium carbonate is readily available and provides a low molecular weight complex and thus requires smaller amounts to provide an amount of calcium relative to organic salts such as calcium gluconate.

For example, co-administered calcium carbonate and vitamin D₂, a sterol that acts synergistically with parathyroid hormone (PTH) to increase the serum calcium concentration, resulted in a decrease in total serum cholesterol in a relatively small subject pool of older women (ages 53-88) (Albanese et al., 1973). However, calcium carbonate and calcium gluconate orally administered as a dietary supplement to men in the age range of 21-26 years old demonstrated a diet-dependent effect (i.e., diets comprised solely of saturated fats or unsaturated fats) on serum cholesterol that rendered the data inconclusive (Bhattacharyya et al., 1969).

Recently, multivariate-adjusted risk analysis was performed on observational data from postmenopausal women and indicated that high levels of calcium supplementation (>1.4 g/day) may lead to reduced risk of ischemic heart disease risk, however milk products proved an inadequate source of calcium (Bostick et al., 1999).

Many of the subject pools were relatively small, however, a larger study involving administering calcium carbonate to 193 subjects with histories of sporadic adenoma demonstrated statistically insignificant changes in total cholesterol, high-density lipoprotein level, and blood pressure (Bostick et al., 2000). Similarly, a statistically insignificant decrease in total cholesterol was observed in a 6 weeks study of 56 subjects administered calcium carbonate (Bell et al., 1992).

Other calcium salts have demonstrated similar inconclusive clinical effects on serum lipids. For example, calcium citrate malate administered in food and as tablets to 13 men (Denke et al., 1993) effected a 6% reduction in plasma cholesterol and a concomitant 7% increase in saturated fat excretion. Low-density lipoprotein levels decreased 11%, but high-density lipoprotein levels increased in some subjects and decreased in others and thus was concluded to be insignificant. Other organic salts (i.e., glycerophosphate and gluconogalactogluconate) have reportedly produced statistically insignificant increases in cholesterol excretion (Mitchell et al., 1968).

High body weight and fat mass have been reported in postmenopausal women with primary hyperparathyroidism (Grey et al., 1994). There is evidence that both parathyroid hormone and 1,25-dihydroxyvitamin D regulate adipocyte activity, the former reducing lipolysis in these cells in vitro, when studied at high concentrations (Zemel et al., 2000; Kelly et al., 1998). Calcium supplementation suppresses circulating concentrations of both parathyroid hormone and 1,25-dihydroxyvitamin D, thereby possibly promoting lipolysis. Studies in transgenic mice with adipocyte expression of the agouti protein, have shown that calcium supplementation increases lipolysis and body temperature, and reduces fatty acid synthase activity and body weight (Zemel et al., 2000). This implies an effect of calcium intake on thermogenesis.

U.S. Pat. No. 6,203,823 teaches a calcium taurate complex as a antihypertensive agent and dietary supplement. This calcium salt is designed for use by individuals with achlorohydria, a condition in which stomach acid cannot be produced, because an acidic environment is not required for calcium bioavailability (i.e., dissociation of the calcium taurate complex advantageously occurs at neutral pH).

Because of the high incidence of cardiovascular disease in postmenopausal women, it is possible that changes in serum lipids resulting from the use of calcium supplements are as important in terms of effects on morbidity and mortality, as its effect on osteoporosis. In light of the unpredictable anecdotal evidence, it is important to clarify whether these lipid changes exist and accurately quantify and characterize those changes in order that the potential health benefits of calcium supplementation is more accurately assessed. The present invention fulfills this need for a population of normal postmenopausal women.

Thus, an inter-relationship between administering calcium citrate to postmenopausal woman and serum lipid levels is not provided in the prior art. The present invention supplies a long-sought solution in the art by providing compositions and methods to improve the health of a postmenopausal woman by increasing high-density lipoprotein levels.

### SUMMARY OF THE INVENTION

One embodiment of the present invention is a method of increasing a high-density lipoprotein level in plasma in a postmenopausal woman comprising the step of administering a pharmaceutical formulation comprising calcium citrate at a therapeutically effective dose. In one preferred embodiment, the high-density lipoprotein level in plasma is increased at least about 5% in the woman, and more preferably, increased at least about 7.7% in the postmenopausal woman.

In another embodiment of the present invention, the therapeutically effective dose of calcium citrate is equivalent to at least about 1 g elemental calcium. In a further embodiment, the pharmaceutical formulation comprises calcium citrate in an amount equivalent to at least about 10 mg elemental calcium, and in a preferred embodiment, at least from about 10 mg to 1 g elemental calcium.

Also contemplated is a pharmaceutical formulation comprising calcium citrate at a therapeutically effective dose and is orally administered to a postmenopausal women for increasing a high-density lipoprotein level in plasma. The pharmaceutical formulation of the present invention is administered for at least about 2 months, and preferably at least about 6 months, and more preferably at least about 12 months.

The present invention further embodies a method of increasing a high-density lipoprotein level in plasma by about 7.7% in a postmenopausal woman comprising the step of administering calcium citrate in an amount equivalent to about 1 g elemental calcium.

In another embodiment is a method of increasing a ratio of high-density lipoprotein to low-density lipoprotein in a postmenopausal woman comprising the step of administering a pharmaceutical formulation comprising calcium citrate at a therapeutically effective dose. In the scope of the present invention, the ratio is increased by about 17% in the postmenopausal woman, and the calcium citrate is in a quantity sufficient to provide at least about 1 g elemental calcium. The pharmaceutical formulation is administered, preferably, by oral ingestion and for at least about 2 months, or preferably for at least about 6 months, and more preferably for at least about 12 months.

In yet a further embodiment, a dietary supplement for a postmenopausal woman is provided that comprises calcium citrate in an amount sufficient to provide at least about 10 mg to about 1 g elemental calcium.

In a specific embodiment, the dietary supplement is provided to the postmenopausal woman for a time sufficient to increase a high-density lipoprotein level in plasma in the woman, and the increase is at least about 5%, and more preferably at least about 7.7%. In further specific embodiments, the dietary supplement is provided for at least about 2 months, or preferably for at least about 6 months, and more preferably for at least about 12 months.

Another embodiment is a method of producing a dietary supplement comprising the step of adding calcium citrate in an amount sufficient to provide about 10 mg to about 1 g elemental calcium to a diet of a postmenopausal woman.

Further, another embodiment is a method of supplementing a diet comprising the step of orally administering calcium citrate in an amount sufficient to provide at least about 10 mg to about 1 g elemental calcium to a postmenopausal woman for a time sufficient to increase a high-density lipoprotein level of the woman.

In a specific embodiment, the high-density lipoprotein level in plasma is preferably increased at least about 5% in the woman, and more preferably increased at least about 7.7% in the woman. In further specific embodiments, administering for at least about 2 months, or preferably for at least about 6 months, and more preferably for at least about 12 months is contemplated.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF SUMMARY OF THE DRAWINGS

The following drawings form part of the present specification and are included to further demonstrate certain aspects of the present invention. The invention may be better understood by reference to one or more of these drawings in combination with the detailed description of specific embodiments presented herein:

FIGS. 1A and 1B Effect of calcium citrate on high-density lipoprotein cholesterol and low-density lipoprotein cholesterol in normal late postmenopausal women.

FIGS. 2A and 2B Effect of calcium citrate on (a) total cholesterol and (b) high-density lipoprotein/low-density lipoprotein ratio in normal late postmenopausal women.

FIG. 3 Effect of calcium citrate or placebo on triglceride concentrations in normal late postmenopausal women. There was no significant treatment effect.

### DETAILED DESCRIPTION OF THE INVENTION

### Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “administering” and “administration” as used herein refers to a mode of delivery and is preferably by oral ingestion. One skilled in the art recognizes that suitable forms of oral ingestion include, but are not limited to, a tablet, a powder, a sustained release tablet, a liquid, a liquid suspension and a capsule. For example, a daily dosage can be divided into one, two and three doses in a suitable form to be administered at one, two and three or more times throughout the day.

The term “dietary supplement” as used herein refers to a compound, either natural or synthetic in origin, that provides nutritional value to the consumer. The nutrient provided for by the supplement is, for example, in an amount recommended for maintaining health (e.g. the RDA amount). The dietary supplement is administered in addition to a regular diet and provides the diet with cumulative amounts of the nutrient in the supplement (i.e., calcium citrate).

The term “medical condition” as used herein is defined as a state of health in which at least one physical trait of an organism is abnormal or deficient. Examples of medical condition include cardiovascular disease, hyperlipidemia and osteoporosis.

The term “therapeutically effective” as used herein is defined as the amount of a compound required to improve some symptom associated with a disease or a medical condition. For example, in the treatment of cardiovascular disease, a compound which decreases, prevents, delays, or arrests any symptom of the disease would be therapeutically effective. A therapeutically effective amount of a compound is not required to cure a disease but will provide a treatment for a disease such that the onset of the disease is delayed, hindered, or prevented, or the disease symptoms are ameliorated, or the term of the disease is changed or, for example, is less severe or recovery is accelerated in the subject.

In this respect, a therapeutically effective amount includes dietary supplemental amounts in that these amount, as taught by the present invention, are critical for delaying, thwarting, and preventing diseases that are not directly associated with their insufficient intake (i.e., below RDA levels). For example, calcium supplementation is known in the art to prevent osteoporosis in women; however, as taught by the present invention, dietary supplemental amounts (i.e., intake amounts in addition to amount provided in a regular diet of the subject) of calcium citrate also effect significant increases in high-density lipoprotein levels in women, which significantly contribute to preventing cardiovascular disease.

The term “pharmaceutically acceptable carrier” as used herein is defined as a molecular entity or composition that does not produce an adverse, allergic or other undesirable reaction when administered to an organism. The carrier includes any and all solvents, dispersion media, coatings, antibacterial or antifungal agents, isotonic and absorption delaying agents and the like. The use of such media and/or agents for pharmaceutical active substances is well known in the art. Except insofar as any conventional media and/or agent is incompatible with the active ingredient, its use in the therapeutic composition is contemplated. Supplementary active ingredients can also be incorporated into the compositions. For human administration, preparations should meet sterility, pyrogenicity, general safety and/or purity standards as required by FDA Office of Biologics standards.

The phrases “pharmaceutically and/or pharmacologically acceptable” refer to molecular entities and compositions that do not produce an adverse, allergic and/or other untoward reaction when administered to an animal or human, as appropriate.

The term “postmenopausal” as used herein refers to the physiological cessation of menses, for at least one year, as a result of decreased ovarian function.

One embodiment of the present invention provides a method of increasing a high-density lipoprotein level in plasma in a postmenopausal woman comprising the step of administering a pharmaceutical formulation comprising calcium citrate at a therapeutically effective dose. A skilled artisan recognizes that plasma lipids refer to lipids, such as cholesterol, circulating in the blood via lipoproteins (i.e., high-density lipoproteins and low-density lipoproteins.), that comprise cholesteryl esters and function as cholesterol carriers through the bloodstream. Lipids include, but are not limited to, triacylglycerols, sphingolipids, phospholipids, and cholesterol. A skilled artisan is aware of methods known in the art to measure lipids and serum lipids found in the plasma of a subject. Non-limiting examples of such analytical methods include colorimetry, enzymatic analysis and fluorometry.

Total serum cholesterol levels of ≦200 mg/dL are considered normal for an adult man or woman and is easily quantitated by methods known in the art. High-density lipoprotein levels are normally 20 to 25% of the total plasma cholesterol and are inversely associated with cardiovascular risk.

Thus, in a preferred embodiment, the high-density lipoprotein level in plasma is increased at least about 5%, and preferably at least about 7.7% in a postmenopausal woman. A skilled artisan is aware of methods to measure high-density lipoprotein cholesterol. As taught herein, low-density lipoprotein levels are empirically derived from total cholesterol and high-density lipoprotein cholesterol levels.

In one specific embodiment of the present invention, the therapeutically effective dose of calcium citrate is equivalent to at least about 1 g elemental calcium. In another specific embodiment, the pharmaceutical formulation comprises calcium citrate in an amount equivalent to at least about 10 mg elemental calcium, and in a preferred embodiment, at least from about 10 mg to 1 g elemental calcium. One of ordinary skill in the art is aware of methods to determine the amount of calcium citrate required to provide an specific amount of elemental calcium, including but not limited, from about 10 mg to about 1 g elemental calcium.

A pharmaceutical formulation comprising calcium citrate at a therapeutically effective dose is also contemplated, and the pharmaceutical formulation is administered to a postmenopausal women for improving a high-density lipoprotein level in plasma. The pharmaceutical formulation is administered for at least about 2 months, and preferably at least about 6 months, and more preferably at least about 12 months.

An alternative embodiment comprises calcium citrate as a dietary supplement that is administered to a postmenopausal woman for improving a high-density lipoprotein level in plasma by about 7.7%.

In a preferred embodiment, the amount of calcium citrate orally administered as a therapeutic agent, either alone or as an adjuvant to one or more pharmaceutical drugs (e.g. an antihypercholesterolemic drug such as lovastatin), is between about 10 mg and about 1 g daily, of elemental calcium. In a particularly preferred embodiment, the amount of calcium citrate administered daily is about 4.1 g, corresponding to about 1 g calcium (i.e., 200 mg above the U.S. RDA). One administration schedule includes oral ingestion of two tablets in the morning and three tablets in the evening although one skilled in the art appreciates that other administration schedules (i.e., three tablets in the morning and two in the evening) are within the scope of the present invention.

The present invention embodies a method of increasing a high-density lipoprotein level in plasma by about 7.7% in a postmenopausal woman comprising the step of administering calcium citrate in an amount equivalent to about 1 g elemental calcium. The step of administering is preferably by oral ingestion and includes administering as a dietary supplement or as a therapeutic agent.

A further embodiment is a method of increasing a ratio of a high-density lipoprotein level to a low-density lipoprotein level in a postmenopausal woman comprising the step of administering a pharmaceutical formulation comprising calcium citrate at a therapeutically effective dose. In the scope of the present invention, the ratio is increased by at least about 17% in the woman, and the calcium citrate is in a quantity sufficient to provide at least about 1 g elemental calcium. The pharmaceutical formulation is preferably administered for at least about 2 months, and preferably for at least about 6 months, and more preferably for at least about 12 months.

The present invention is based on the discovery that calcium citrate increases the level of high density lipoproteins and thus, selectively reduces the ratio of high-density lipoprotein to low density lipoproteins in postmenopausal woman. The woman may or may not demonstrate an abnormal ratio of high-density lipoprotein to low-density lipoprotein level, in which the ratio is normally in the range of about 0.3 to about 0.6. For example, if a postmenopausal woman demonstrates an abnormal ratio, then the pharmaceutical formulation of the present invention is orally administered to the woman in a single or multiple dose administration regimen in an amount of calcium citrate sufficient to increase the abnormal ratio.

A skilled artisan is aware that it an abnormal high-density to low-density lipoprotein ratio increases the risk of conditions such as myocardial infarction, cardiac ischemia, cerebral ischemia and peripheral vascular diseases, not the mere presence of high levels of cholesterol (see U.S. Pat. No. 4,255,449) However, if a postmenopausal woman does not demonstrate an abnormal ratio, then a dietary supplement of the present invention is orally administered to the woman in a single or multiple dose administration regimen in an amount of calcium citrate sufficient to maintain a normal high-density to low-density lipoprotein ratio by maintaining or increasing the high-density lipoprotein level of the woman. In such an example, the woman further increases the normal ratio within the range of a healthy woman and further substantially reduces her risk of developing cardiovascular disease.

In yet a further embodiment, a dietary supplement for a postmenopausal woman is provided that comprises calcium citrate in an amount sufficient to provide at least about 10 mg to about 1 g elemental calcium. In a non-limiting example, a chronic diet substantially supplemented with calcium citrate is orally administered to a postmenopausal woman to increase high-density lipoprotein levels of the woman.

In alternative embodiments, the supplement comprises composition foods having necessary vitamins and minerals for sustained life, and the calcium citrate is administered to extend life by increasing the high-density lipoprotein level of the postmenopausal woman. Non-limiting examples of forms of dietary supplements suitable for oral administration with vitamins and minerals include powders, tablets, liquids, liquid suspensions and capsules.

In specific embodiments, the dietary supplement is provided to the postmenopausal woman for a time sufficient to increase a high-density lipoprotein level in plasma in the woman, and the increase is at least about 5%, and more preferably at least about 7.7%. In further specific embodiments, the dietary supplement is provided for at least about 2 months, or preferably for at least about 6 months, and more preferably for at least about 12 months.

Another embodiment is a method of producing a dietary supplement comprising the step of adding calcium citrate in an amount sufficient to provide about 10 mg to about 1 g elemental calcium to a diet of a postmenopausal woman. A person of ordinary skill in the art recognizes that methods to prepare pharmaceutical formulations are suitable for preparation of dietary supplements that are administered in a similar manner (i.e., oral ingestion).

Further, another embodiment is a method of supplementing a diet comprising the step of orally administering calcium citrate in an amount sufficient to provide at least about 10 mg to about 1 g elemental calcium to a postmenopausal woman in order to effect an increase in a high-density lipoprotein level of the woman.

In a specific embodiment, the high-density lipoprotein level in plasma is increased at least about 5% in the woman, and preferably increased at least about 7.7% in the woman. In further specific embodiments, administering for at least about 2 months, or preferably for at least about 6 months, and more preferably for at least about 12 months is contemplated.

The calcium citrate of the present invention is substantially pure for ready formulation into a desired vehicle, where appropriate. It must be stable under the conditions of manufacture and storage and must be preserved against the contaminating action of microorganisms, such as bacteria and fungi, and it can be formulated into a composition in a neutral or salt form. Examples of pharmaceutically acceptable salts, include but are not limited to, calcium citrate tetrahydrate and calcium citrate tribasic tetrahydrate. Calcium citrate may be formulated within a therapeutic mixture to comprise about 10 milligrams, or about 100 milligrams, or about 200-500 milligrams or even about 1 gram elemental calcium per dose and so. Multiple doses can also be administered.

Aqueous compositions of the present invention comprise an effective amount of calcium citrate, dissolved or dispersed in an aqueous medium or a pharmaceutically acceptable carrier. Aqueous compositions of calcium citrate are also contemplated, and may contain the calcium citrate in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin. One such example of an aqueous composition comprising calcium citrate is a drink formulated to provide calcium citrate in a therapeutically effective amount.

Solutions of the active compound as a free base or a pharmacologically acceptable salts are prepared in water suitably mixed with a surfactant, such as hydroxypropylcellulose. Dispersions are prepared in glycerol, liquid polyethylene glycols, and/or mixtures thereof and/or in oils. Under ordinary conditions of storage and/or use, these preparations contain a preservative to prevent the growth of microorganisms. A carrier can also be a solvent or dispersion medium containing, for example, water, ethanol, polyol (for example, glycerol, propylene glycol, and/or liquid polyethylene glycol, and the like), suitable mixtures thereof, and vegetable oils. The proper fluidity can be maintained, for example, by the use of a coating, such as lecithin, by the maintenance of the required particle size in the case of dispersion and/or by the use of surfactants. The prevention of the action of microorganisms can be brought about by various antibacterial or antifungal agents, for example, parabens, chlorobutanol, phenol, sorbic acid, thimerosal, and the like.

Formulations are administered in a manner compatible with the dosage formulation and/or in such amount as is therapeutically effective. The formulations are easily administered in a variety of dosage forms, such as the type of injectable solutions described above, but sustained-release capsules and/or the like can also be employed.

A preferred method of delivery includes oral administration and thus requires oral formulations. Pharmaceutically acceptable forms include, e.g., tablets and other solids for oral administration; liposomal formulations; time release capsules; liquids such as a drink mixture, liquid suspensions and any other form currently used. In addition, antimicrobial preservatives, if required, may be included in the formulation. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations and powders, and may be prepared according to any method known in the art for the manufacture of pharmaceutical compositions.

In certain defined embodiments, oral pharmaceutical compositions comprise an inert diluent and assimilable edible carrier, or they may be enclosed in hard or soft shell gelatin capsule, or they may be compressed into tablets, or they may be a powder, or they may be incorporated directly with the food of the diet such as a snack bar or a drink. For oral therapeutic administration, an active compound is incorporated with excipients and used in the form of ingestible tablets, buccal tables, troches, capsules, elixirs, suspensions, syrups, liquids, wafers, and the like. For example, the composition may comprise a soft gelatin capsules wherein the active ingredient is mixed with water or an oil medium, such as peanut oil, liquid paraffin or olive oil. The percentage of calcium citrate in the compositions and preparations may, of course, be varied and may conveniently be between about 2 to about 90% of the weight of the unit, and preferably between 25-90%. The amount of active compounds in such therapeutically useful compositions is such that a suitable dosage will be obtained.

Tablets may be uncoated or coated by known techniques to delay disintegration and absorption in the gastrointestinal tract and thereby provide a sustained release and sustained action over a longer period of time. For example, a time delay material such as glyceryl monostearate or glyceryl distearate alone or with a wax may be employed.

The tablets, troches, pills, capsules and the like may also contain the following: a binder, as gum tragacanth, acacia, cornstarch, and gelatin; excipients, such as dicalcium phosphate; a disintegrating agent, such as corn starch, potato starch, alginic acid and the like; a lubricant, such as magnesium stearate; and a sweetening agent, such as sucrose, lactose and saccharin may be added and a flavoring agent, such as peppermint, oil of wintergreen, and cherry flavoring. The sweetening and flavoring agents increases the palatability of the preparation. When the dosage unit form is a capsule, it may contain, in addition to materials of the above type, a liquid carrier. Various other materials may be present as coatings and to otherwise modify the physical form of the dosage unit. For instance, tablets, pills, and capsules may be coated with shellac, sugar or both. A syrup of elixir may contain the active compounds sucrose as a sweetening agent methyl and propylparabens as preservatives, a dye and flavoring, such as cherry or orange flavor.

Although oral administration is preferred, a skilled artisan recognizes other possible delivery mechanisms such as an intravenous formulation providing a dietary supplement or a therapeutic agent such as described herein. Manufacturing processes for formulations suitable for various other delivery mechanisms are well known in the art.

### EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Method of Subject Selection

A randomized controlled trial of calcium supplementation in 223 normal postmenopausal women aged 55 years or older. Women receiving therapy for osteoporosis were ineligible, as were those with any other major ongoing disease, including serum creatine greater than 0.2 mmol/L, untreated hypo- or hyperthyroidism, liver disease, serum 25-hydroxyvitamin D less than 10 μg/L, malignancy or metabolic bone disease. None of the subjects had been regular users of hormone replacement therapy, anabolic steroids, glucocorticoids or bisphosphonates in the previous 1 year. Lumbar spine bone density (Lunar Expert, Madison, Wis.) was not below the age-appropriate normal range. In the present sub-study there was the further requirement that subjects were not receiving therapy for hyperlipidemia.

266 subjects were assessed for eligibility for the sub-study, and 223 entered the study, of which 111 women were randomized to calcium and 112 to placebo. 20 women in the calcium group discontinued study medication as did 26 in the placebo group. Baseline clinical data for the subject groups is shown in Table 1. The groups were comparable in all indices at baseline. The study was approved by the local ethics committee, and each subject gave written, informed consent. There were not significant differences between groups.

TABLE 1
Clinical Characteristics of Study Subjects

| Characteristic | Placebo | Calcium |
|---|---|---|
| N | 112 | 111 |
| Age (year) | 72 (4) | 72 (4) |
| Years since menopause | 22 (6) | 22 (6) |
| Weight (kg) | 68 (11) | 66 (10) |
| Height (m) | 159 (6) | 159 (5) |
| Physical activity | 33 (4) | 34 (5) |
| (kcal/kg/day) | | |
| Calcium intake (mg/d) | 910 (400) | 910 (440) |
| Number of smokers | 4 | 4 |
| Compliance | 74 (27) | 80 (21) |
| Date are mean (std dev). | | |

### Example 2

### Method of Treatment

Treatments were allocated randomly using a minimization algorithm balancing for current thiazide use, age and the occurrence of fractures resulting from minimal trauma after the age of 40 years. Subjects received 1 gram of elemental calcium daily as the citrate (Citracal, Mission Pharmacal, San Antonio Tex.) or identical placebo. They were asked to take two tablets (each containing 200 mg elemental calcium) before breakfast and three tablets in the evening. Calcium intake and physical activity were assessed using validated questionnaires (Angus et al., 1989; Wilson et al., 1986). Compliance was assessed by tablet counts.

### Example 3

### Lipid Measurements

Fasting total cholesterol, high-density lipoprotein cholesterol and triglycerides were measured in serum at baseline, 2 months, 6 months, and one year, using a Roche-Hitachi 747 autoanalyzer (Mannherim, Germany). The cholesterol assay used cholesterol oxidase, high-density lipoprotein measurement was by a direct non-precipitation method using PEG-modified enzymes (cholesterol oxidase), and triglycerides were assayed using a glycerol kinase/oxidase method after initial hydrolysis using lipoprotein lipase. Low-density lipoprotein was calculated using the Friedewald formula:

LDL=TC−HDL−(0.45×triglyceride)

where,

LDL=low-density lipoprotein level

TC=total cholesterol level

HDL=high-density lipoprotein level

Baseline lipid data for the subject groups is shown in Table 2. The groups were statistically comparable in all indices at baseline.

TABLE 2
Baseline Fasting Serum Lipid Concentrations

| Index | Placebo | Calcium |
|---|---|---|
| Total Cholesterol (mmol/L) | 6.56 (1.04) | 6.72 (1.20) |
| HDL Cholesterol (mmol/L) | 1.59 (0.40) | 1.65 (0.45) |
| LDL Cholesterol (mmol/L) | 4.26 (0.98) | 4.39 (1.16) |
| HDL/LDL | 0.40 (0.17) | 0.42 (0.19) |
| Triglycerides (mmol/L) | 1.57 (0.763) | 1.55 (0.83) |
| Date are mean (std dev). | | |

### Example 4

### Statistical Evaluation

The limited earlier studies (Yacowitz et al., 1965; Carlson et al., 1971; Albanese et al., 1973) suggested that calcium supplementation might produce a mean (SD) change in serum total cholesterol of −0.5 (0.6) mmol/L. This indicated that 108 subjects would be necessary for the study to have a power of 90% when ∝=0.01. In light of the uncertainty of the likely size of effect to be expected, and to allow power to assess effects on cholesterol sub-fractions and triglycerides, a study size of twice this number was adopted.

Data were analyzed on an intention-to-treat basis, using a fixed effects mixed models approach to repeated measures using the MIXED procedure of SAS (SAS Institute, Cary, N.C.). Significant main and interaction effects were explained using the method of Tukey. In complimentary analyses, the change from baseline for each continuous normal dependant variable was compared between treatment and placebo arms using Student's t-test for independent groups. A 5% significance level was maintained throughout these analyses.

### Example 5

### Intention to Treat Analysis

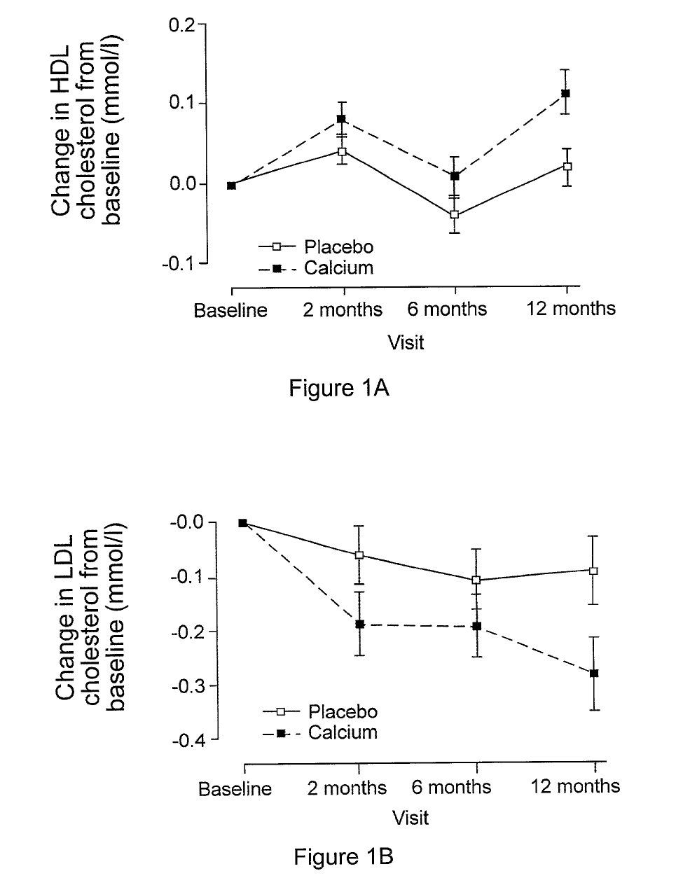

The effect of calcium citrate on high-density lipoprotein cholesterol (FIG. 1A) and low-density lipoprotein cholesterol (FIG. 1B) in normal late postmenopausal women were determined. There were significant treatment effects on both parameters (P=0.05 and P=0.04, respectively). Data are changes from baseline, and are shown as mean with standard errors.

In the calcium group, high-density lipoprotein increased 7.3% above baseline at 12 months (comparison with placebo group, P=0.058). In contrast, low-density lipoprotein declined 7.8% over this time in those taking calcium citrate (comparison with placebo group, P=0.079). As a result, total cholesterol showed a non-significant fall of 3.7%, and the high-density lipoprotein/low-density lipoprotein ratio increased 17.7% in the calcium group (P<0.0001) in comparison with a 4.2% increase in this ratio in the placebo subjects (P>0.10). The between groups comparison for changes in the high-density lipoprotein/low-density lipoprotein ratio was significant (P=0.002). Re-expressing the data as the ratio of total cholesterol/high-density lipoprotein there was a decline of 9.3% in the calcium group at one year (P<0.0001) in comparison with a fall of 3.5% in the placebo group (P=0.02). The between-groups difference was again significant (P=0.028), but there was no significant treatment effect on triglycerides (P=0.48).

Body weight (mean ±SE) decreased by 289±193 g in the calcium group and by 91±255 g in those taking placebo. Neither of these changes was significant, and there was no between-groups effect on body weight (P=0.54).

### Example 6

### Protocol Analysis

No subjects were taking lipid lowering drugs at study entry but 4 women in the placebo group and 3 in the calcium group commenced either a statin or a fibrate during the study period. Since the effects of these agents on the study end-points are likely to be far greater than those of calcium, the analysis was repeated after the exclusion of these subjects which are shown in FIGS. 1-3.

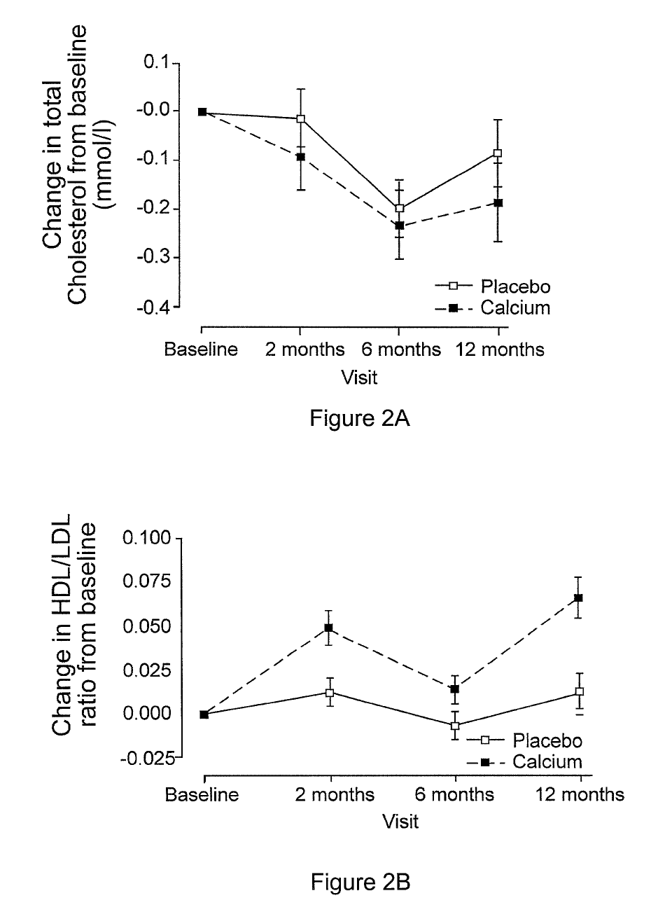

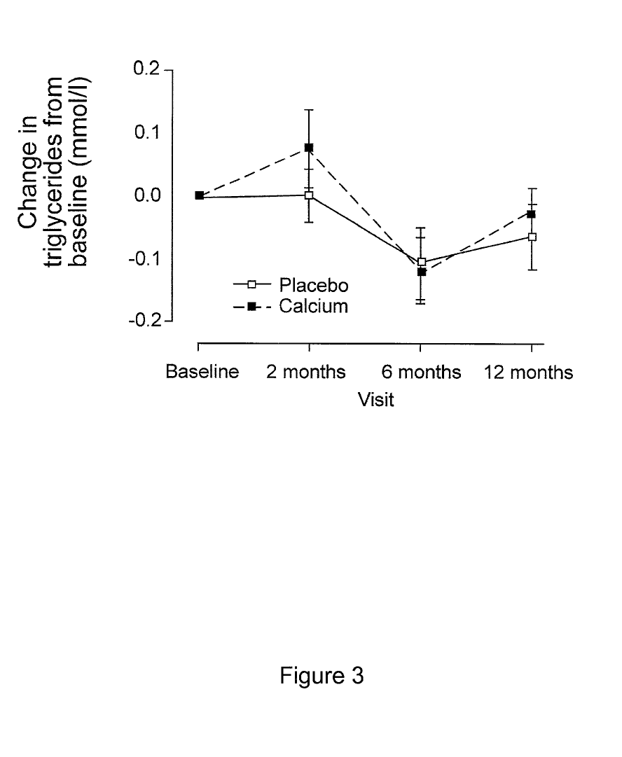

The effect of calcium citrate on total cholesterol (FIG. 2A) and high-density lipoprotein/low-density lipoprotein ratio (FIG. 2B) in normal late postmenopausal women were determined. There was insignificant change in total cholesterol levels, but high-density lipoprotein/low-density lipoprotein increased significantly more in those receiving calcium than placebo (P=0.0015). Data are changes from baseline, and shown as mean with standard errors. As indicated, there was an increase in high-density lipoprotein in those taking calcium which was significantly different from the changes in the placebo group (P=0.05, FIG. 1A). Low-density lipoprotein declined in the calcium group (FIG. 1B, P=0.04 in comparison with placebo. Thus, total cholesterol did not change significantly (FIG. 2A) but the high-density lipoprotein/low-density lipoprotein ratio increased 17% (FIG. 2B, P−0.0015) and the total cholesterol/high-density lipoprotein ratio decreased by 9.6% (between-groups comparison, P=0.03). There was no significant treatment effect on triglycerides (FIG. 3).

### Example 7

### Effects of Calcium Citrate on Serum Lipids

The present data indicate that the use of calcium citrate in a daily dose comprising about 1 gram of elemental calcium, increased serum high-density lipoprotein cholesterol with reciprocal changes in low-density lipoprotein cholesterol. As a consequence, the high-density lipoprotein/low-density lipoprotein ratio increases by almost 20%. These unexpected changes in high-density lipoprotein are only one fifth of those seen with statins (Scandinavian Simvastatin Survival Study Group, 1994). The changes in total cholesterol/high-density lipoprotein are about one third of the statin-induced effect. On this basis, the effects of calcium on lipids would be predicted to reduce cardiovascular event rates by 20-30%. Because many postmenopausal women develop cardiovascular disease, the hypolipidemic effect of calcium demonstrated in the present invention are likely to have a greater effect on both morbidity and mortality in postmenopausal women than its effects on osteoporosis.

### Example 8

### Weight Loss and Calcium Citrate

Weight loss was not observed to be statistically significant, however the between-groups differences in weight were comparable in size and direction with those recently reported as a significant effect of calcium on weight loss (Davies et al., 2000). Further, calcium is capable of complexing lipids in the gut, and this ability is considered to contribute to a direct adipocyte effect that are suggested to contribute to weight loss during periods of calcium supplementation. Because postmenopausal women in primary hyperparathyroidism have reportedly high body weight and fat mass, calcium supplementation to effect weight loss in postmenopausal women is plausible. Thus, calcium intake effects a complex control mechanism on intermediary metabolism resulting in beneficial effects not only on high-density lipoprotein levels, and consequently, the development of cardiovascular disease, but also potentially on body weight.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### Patents

U.S. Pat. No. 6,203,823

U.S. Pat. No. 4,255,449

Genant H. K., Cooper C., Poor G., Reid I., Ehrlich G., Kanis J. Nordin B E C, Barrett-Connor E., Black D., Bonjour J. P., Dawson-Hughes B., Delmar P. D., Dequeker J., Eis S. R., Gennari C., Johnell O., Johnston C. C., Lau E. M. C., Liberman U. A., Lindsay, R., Martin T. J., Masri B., Mautalen C. A. Meunier P. J., Miller P. D., et al. Interim report and recommendations of the World Health Organization task-force for osteoporosis. Osteoporosis Int. 1999; 10: 259-264.

Dawson-Hughes B., Dallal G. E., Krall, E. A., Sadowski, L., Sahyoun, N., Tannenbaum, S. A. controlled trial of the effect of calcium supplementation on bone density in postmenopausal women. N. Engl. J. Med. 1990; 323: 878-883.

Reid I. R., Ames R. W., Evans M. C. Gamble, G. D., Sharpe, S. J. Effect of calcium supplementation on bone loss in postmenopausal women. N. Engl. J. Med. 1993; 328: 460-464.

Chevalley T., Rizzoli, R., Nydegger, V., Slosman, D., Rapin, C. H., Michel, J. P., Vasey H., Bonjour J. P. Effects of calcium supplements on femoral bone mineral density and vertebral fracture rate in vitamin-D-replete elder patients. Osteoporosis Int. 1994; 4: 245-252.

Reid I. R., Ames R. W., Evans M. C., Gamble G. D., Sharpe S. J. Long-term effects of calcium supplementation on bone loss and fractures in postmenopausal women—a randomized controlled trial. Am. J. Med. 1995; 98: 331-334.

Recker R. R., Hinders S., Davies, K. M., Heaney R. P., Stegman, M. R., Lappe, J. M., Kimmel, D. B. Correcting calcium nutritional deficiency prevents spine fractures in elderly women. J. Bone Miner Res. 1996; 11: 1961-1966.

Bargerlux M. J., Heaney R. P. The role of calcium intake in preventing bone fragility, hypertension, and certain cancers. J. Nutr. 1994; 124: S1406-S1411.

Govers, M. J. A. P., Vandermeer R. Effects of Dietary Calcium and Phosphate on the Intestinal Interactions Between Calcium, Phosphate, Fatty Acids, and Bile Acids. Gur 1993; 34: 365-370.

Danke M. A., Fox M. M., Schulte M. C. Short-term dietary calcium fortification increases fecal saturated fat content and reduces serum lipids in men. J. Nutr. 1993; 123: 1047-1053.

Mitchell W. D., Fyfe T., Smith, D. A. The effect of oral calcium on cholesterol metabolism. J. Atherosclerosis Res. 1968; 8: 913-922.

Yacowitz H., Fleischman, A. I., Bierenbaum, M. L., Effects of oral calcium on serum lipids in man. B. M. J. 1965; 1: 1352-1354.

Carlson, L. A., Olsson, A. g., Oro L., Rossner S. Effects of oral calcium on serum cholesterol and triglycerides in patients with hyperlipidemia. Atherosclerosis 1971; 14: 391-400.

Albanese A. A., Edelson A. H., Woodhull, M. L., Lorenze, E. J., Wein, E. H., Orto, L. A. Effect of a calcium supplement on serum cholesterol, calcium, phosphorus and bone density of normal healthy elderly females. Nutr. Rep. Int. 1973; 8: 119-130.

Bierenbaum, M. L., Fleischman, A. I., Raichelson, R. I. Long-term human studies on he lipid effects of oral calcium. Lipids 1972; 7; 202-206.

Bell L. Halstenson C. E., Halstenson, D. J., Macres, M., Keane, W. F. Cholesterol-lowering effects of calcium carbonate in patients with mild to moderate hypercholesterolemia. Arch Int. Med. 1992; 152: 2441-2444.

Bostick R. M., Fosdick L., Grandits, G. A., Grambsch, P., Gross M., Louis, T. A. Effect of calcium supplementation on serum cholesterol and blood pressure. A randomized, double-blind, placebo-controlled, clinical trial. Archives of Family Medicine 2000; 9: 31-38.

Angus R. M., Sambrook, P. N., Pocock, N. A., Eisman, J. A. A simple method for assessing calcium intake in Caucasian women. J. Am. Diet Assoc. 1989; 89: 209-214.

Wilson, P. W. F., Paffenbarger, R. S., Morris, J. N., Havlik, R. J. Assessment methods for physical fitness activity in population studies: report of a N.H.L.B.I. workshop. Am. Heart, J. 19086; 111: 1177-1192.

Scandinavian Simvastatin Survival Study Group, Randomised trial of cholesterol lowering in 4444 patients with coronary heart disease: the Scandinavian Simvastatin Survival Study (4S). Lancet 1994; 344: 1383-1389.

Barr, S. T., McCarron, D. A., Heaney, R. P., Dawson-Hughes, B., Berga, S. L., Stem, J. S., Oparil S. Effects of increased consumption of fluid milk on energy and nutrient intake, body weight, and cardiovascular risk factors in healthy older adults. J. Am. Diet Assoc. 2000; 100: 810-817.

Bostick, R. M., Kushi, L. H., Wu Y., Meyer, K. A., Sellers, T. A., Folsom A. R. Relation of calcium, vitamin D and dairy food intake to ischemic heart disease mortality among postmenopausal women. Am. J. Epidemiol 1999; 149: 151-161.

Ness, A. R., Smith, G. D., Hart, C. Milk, coronary heart disease and mortality. Journal of Epidemiology & Community Health 2001; 55: 379-382.

Zemel, M. B., Shi H., Greer B. Dirienzo, D., Zemel, P. C. Regulation of adiposity by dietary calcium. FASEB Journal 2000; 14: 1132-1138.

Kelly, K. A., Gimble, J. M. 1,25-dihydroxy vitamin d-3 inhibits adipocyte differentiation and gene expression in murine bone marrow stromal cell clones and primary cultures. Endocrinology 1998; 139: 2622-2628.

Grey, A. B., Evans, M. C., Stapleton, J. P., Reid, I. R. Body weight and bone mineral density in postmenopausal women with primary hyperparathyrodism. Ann. Intern. Med. 1994; 121: 745-749.

Davies, K. M., Heaney, R. P., Recker, R. R., Lappe, J. M., Barger-Lux, M. J., Rafferty, K., Hinders, S. Calcium intake and body weight. J. Clin. Endocrinol. Metab. 2000; 85: 4635-4638.

McCarron, D. A., Morris, C. D., Henry, H. J., Stanton, J. L. Blood pressure and nutrient intake in the United States. Science 1984; 224: 1392-1398. “Remington's Pharmaceutical Sciences” 15th Edition, pages 1035-1038, 1570-1580.

We claim:

1. A method of increasing a high-density lipoprotein level in plasma in a postmenopausal woman comprising the step of administering a pharmaceutical formulation comprising calcium citrate at a therapeutically effective dose.

2. The method of claim 1, wherein the high-density lipoprotein level in plasma is increased at least about 5% in said woman.

3. The method of claim 1, wherein the high-density lipoprotein level in plasma is increased at least about 7.7% in said woman.

4. The method of claim 1, wherein the therapeutically effective dose of calcium citrate is equivalent to at least about 1 g elemental calcium.

5. The method of claim 1, wherein the pharmaceutical formulation comprises calcium citrate in an amount equivalent to at least about 10 mg elemental calcium.

6. The method of claim 1, wherein the pharmaceutical formulation comprises calcium citrate in an amount equivalent to at least from about 10 mg to 1 g elemental calcium.

7. The method of claim 1, wherein said administering is for at least about 2 months.

8. The method of claim 1, wherein said administering is for at least about 6 months.

9. The method of claim 1, wherein said administering is for at least about 12 months.

10. A pharmaceutical formulation for oral administration comprising calcium citrate at a therapeutically effective dose, wherein said formulation is administered to postmenopausal women for increasing a high-density lipoprotein level in plasma.

11. A method of increasing a high-density lipoprotein level in plasma by about 7.7% in a postmenopausal woman comprising the step of administering calcium citrate in an amount equivalent to about 1 g elemental calcium.

12. A method of increasing a ratio of high-density lipoprotein to low-density lipoprotein in a postmenopausal woman comprising the step of administering a pharmaceutical formulation comprising calcium citrate at a therapeutically effective dose.

13. The method of claim 13, wherein said ratio is increased by at least about 17% in said woman.

14. The method of claim 13, wherein said calcium citrate is in a quantity sufficient to provide at least about 1 g elemental calcium.

15. The method of claim 13, wherein said administering is for at least about 2 months.

16. A dietary supplement for a postmenopausal woman comprising calcium citrate in an amount sufficient to provide at least about 10 mg to about 1 g elemental calcium.

17. A method of producing a dietary supplement comprising the step of adding calcium citrate in an amount sufficient to provide about 10 mg to about 1 g elemental calcium to a diet of a postmenopausal woman.

18. A method of supplementing a diet comprising the step of orally administering calcium citrate in an amount sufficient to provide at least about 10 mg to about 1 g elemental calcium to a postmenopausal woman, wherein said administering increases a high-density lipoprotein level in plasma of said woman.

19. The method of claim 18, wherein the high-density lipoprotein level in plasma is increased at least about 5% in said woman.

20. The method of claim 18, wherein the high-density lipoprotein level in plasma is increased at least about 7.7% in said woman.

21. The method of claim 18, further comprising administering for at least about 12 months.
